(19) United States
(12) Patent Application Publication
(10) Pub. No.: US 2026/0209677 A1
(43) Pub. Date: Jul. 23, 2026

(54) Reduction Of The CO2 Concentration In A CO2 Incubator

(71) Applicant: Thermo Electron LED GmbH, Langenselbold (DE)

(72) Inventors: Jürgen Schneider, Langenselbold (DE); Tino Barnkoth, Langenselbold (DE); Herr Holger Aulbach, Langenselbold (DE)

(21) Appl. No.: 19/455,107

(22) Filed: Jan. 21, 2026

(30) Foreign Application Priority Data
Jan. 23, 2025 (DE) 102025102470.7

(51) Int. Cl. C12M 1/00; B01D 46/54; C12M 1/34

(52) U.S. Cl. CPC C12M 41/14; B01D 46/543; C12M 29/00; C12M 29/04; C12M 41/34

(57) ABSTRACT

The invention relates to a CO₂ incubator comprising a usable space, a pressure equalization opening that is designed to adjust the pressure in the usable space to the ambient pressure, and a CO₂ supply, wherein the CO₂ incubator is designed to reduce any CO₂ concentration in the usable space. The invention also relates to a corresponding method for changing the CO₂ concentration in the usable space of a CO₂ incubator.

The present invention relates to the field of CO₂ incubators, and in particular the incubation of cells in cell culture and cell therapy applications using large containers for the cultivation of a large number of cells in an incubator.

In CO₂ incubators, cells can be stored in large containers (e.g. G-Rex® containers) during their growth phase, particularly in cell culture and cell therapy applications. The living cells release CO₂ during growth. With a large number of cells, which is typical for the applications stated above, the CO₂ release can lead to a significant increase in the CO₂ concentration within the CO₂ incubator, with the result that this may exceed the actual setpoint. This increase cannot typically be offset by known CO₂ incubators, meaning that the CO₂ concentration and therefore the pH value in the medium may undergo a critical change. This can lead to damage or, in a worst case scenario, a complete loss of the cells.

Known CO₂ incubators do not usually have the option of reducing the CO₂ concentration in a controlled manner. Specifically, although the CO₂ control system can add CO₂ by introducing CO₂ gas into the device, an apparatus for controlled reduction of the CO₂ concentration within the device is not typically available. Users can indeed open the door of the CO₂ incubator in order to reduce the CO₂ concentration. The drawback with this, however, is that the temperature and humidity also approach the values of the atmosphere surrounding the device and deviate therefore from the corresponding setpoints, at least temporarily.

Against this background, one object of the present invention is to overcome or at least reduce the shortcomings and drawbacks associated with the state of the art. In general, the object of the present invention may be to enable an active reduction of the CO₂ concentration within the CO₂ incubator.

This object is achieved through the subject matter of the independent patent claims. Beneficial developments of the invention are described in the dependent patent claims, the description that follows, and the figures.

According to a first aspect, the invention relates to a CO₂ incubator comprising a usable space, a pressure equalization opening that is designed to adjust the pressure in the usable space to the ambient pressure, and a CO₂ supply, wherein the CO₂ incubator is designed to reduce any CO₂ concentration in the usable space.

The CO₂ incubator according to the invention can therefore reduce the CO₂ concentration in the usable space in particular. This has the advantage of preventing an unwanted increase in the CO₂ concentration in the usable space. A corresponding increase can for example be caused by a large number of cells that release CO₂ during their growth.

In embodiments of the invention, the incubator may be designed to supply controlled air to the usable space.

In embodiments of the invention, the incubator may be designed to actively introduce supply air into the usable space.

Alternatively, in embodiments of the invention, the incubator may be designed to actively extract air from the usable space and as a result feed supply air via the pressure equalization opening.

In embodiments of the invention, the pressure equalization opening may comprise a pressure equalization filter, which preferably comprises a sintered filter.

In embodiments of the invention, the incubator may comprise a pump.

In embodiments of the invention, the pump may be designed to extract air from the usable space.

It is understood that the term “air” in this context refers to the gas and/or aerosol mixture that is present in the usable space.

In embodiments of the invention, an inlet of the pump may be fluidically connected to the usable space.

In embodiments of the invention in which the incubator is designed to actively extract air from the usable space, the incubator may comprise an air outlet valve which is fluidically connected to the usable space and an air outlet reservoir, and be arranged between these, wherein the air outlet reservoir has a lower pressure than the usable space.

In embodiments of the invention, the pump may be designed to feed supply air into the usable space.

In embodiments of the invention, an outlet of the pump may be fluidically connected to the usable space.

In embodiments of the invention, the incubator may further comprise a supply air filter arranged downstream of the pump.

In embodiments of the invention, an inlet of the pump may be connected to a supply air reservoir.

In embodiments of the invention, the incubator may comprise an air duct, wherein the air duct comprises:
an inlet and an outlet, which are in each case connected to the usable space, a fan designed to convey air from the inlet to the outlet of the air duct, and
a flow resistance arranged upstream of the fan and downstream of the inlet;
wherein a supply air duct connected to a supply air reservoir is arranged between the flow resistance and the fan (52).

It may also be provided that the air duct is designed in such a way that a negative pressure can be generated between the flow resistance and the fan. The negative pressure may be at least 0.5 mbar, preferably at least up to 1 mbar below atmospheric pressure.

In embodiments of the invention, the flow resistance may be an air duct filter, preferably a particulate filter, and more preferably a HEPA filter.

In embodiments of the invention, a supply air filter may be arranged in the supply air duct.

In embodiments of the invention, a supply air valve may be arranged in the supply air duct.

In embodiments of the invention, the supply air valve may be arranged downstream of the supply air filter.

In embodiments of the invention, the supply air valve may be a proportional valve.

In embodiments of the invention, the air duct may comprise a humidifier arranged upstream of the flow resistance.

In embodiments of the invention, the incubator may comprise a CO₂ sensor designed to determine the CO₂ concentration in the usable space.

### In Embodiments of the Invention, the Incubator May Comprise a Controller.

In embodiments of the invention, the controller may be designed to reduce the CO₂ concentration in the usable space if a CO₂ limit value is exceeded.

In embodiments of the invention, the controller may be designed to control and/or regulate the CO₂ concentration in the usable space with respect to a predetermined setpoint.

In embodiments of the invention, the controller may be designed to control and/or regulate the supply air valve and as an option also the fan.

In embodiments of the invention, the pump may be an air pump or a vacuum pump
In embodiments of the invention, the controller may be designed to control and/or regulate the pump.

In embodiments of the invention, the supply air filter may be a membrane filter.

In embodiments of the invention, the supply air filter may be a sterile filter, preferably made of PTFE.

In embodiments of the invention, the CO₂ supply may comprise a CO₂ valve and a CO₂ filter and be designed to be connected to a CO₂ reservoir.

In embodiments of the invention, the CO₂ filter may be arranged upstream of the CO₂ valve.

In embodiments of the invention, the CO₂ valve may be a proportional valve.

In embodiments of the invention, the incubator may be designed in such a way that the pump does not operate if the CO₂ valve is open.

In embodiments of the invention, the CO₂ filter may be a membrane filter.

In embodiments of the invention, the CO₂ filter may be a sterile filter, preferably made of PTFE.

In embodiments of the invention, the supply air may be at least one of circulating air from the incubator, oxygen and/or nitrogen.

In embodiments of the invention, the supply air reservoir may be provided by the environment of the CO₂ incubator.

According to a further aspect, the invention is targeted at a method for changing the CO₂ concentration in the usable space of a CO₂ incubator, wherein the method comprises: Determination of the CO₂ concentration in the usable space, comparison of the CO₂ concentration with a predetermined CO₂ value, reduction of the CO₂ concentration in the usable space of the CO₂ incubator if the CO₂ value is exceeded.

In embodiments of the invention, the predetermined CO₂ value may be a CO₂ limit value.

In embodiments of the invention, the predetermined CO₂ value may be a CO₂ setpoint.

In embodiments of the invention, the determination of the CO₂ concentration may comprise measurement of the CO₂ concentration in the air within the CO₂ incubator.

In embodiments of the invention, the reduction of the CO₂ concentration in the usable space may comprise the active delivery of supply air to the usable space.

In embodiments of the invention, the active delivery of supply air may comprise the extraction of air from the usable space in order to thereby allow supply air to enter, for example via a pressure equalisation opening.

In embodiments of the invention, the active delivery of supply air may comprise pumping supply air into the usable space.

In embodiments of the invention, the active delivery of supply air may comprise the suction of supply air via a supply air duct connected to an air duct of the incubator via negative pressure.

In embodiments of the invention, the method may further comprise filtering of the supply air.

In embodiments of the invention, the supply air may be fed from a supply air reservoir.

The supply air reservoir may for example be the surrounding environment of the CO₂ incubator, with the result that the supply air corresponds to the ambient air. Alternatively, the supply air reservoir may also be provided by a container, e.g. a gas cylinder, meaning that the composition of the supply air can be controlled precisely.

In embodiments of the invention, the method may further comprise: Increasing the CO₂ concentration in the usable space of the CO₂ incubator if the CO₂ value falls below the setpoint.

In embodiments of the invention, the method may involve receiving the CO₂ value.

In embodiments of the invention, the method may comprise control or regulation of the CO₂ concentration as a function of the CO₂ value.

In embodiments of the invention, the regulation of the CO₂ concentration may involve determining, comparing, increasing, and reducing the CO₂ concentration in the usable space.

In embodiments of the invention, the CO₂ incubator may be a CO₂ incubator according to one of the incubator embodiments described above.

In embodiments of the invention, the incubator may be designed to implement the method according to one of the method embodiments described above.

In embodiments of the invention, the controller may be designed to implement the method according to one of the method embodiments described above.

The invention is also defined by the following numbered embodiments.

Reference is made below to incubator embodiments. These embodiments are identified by a letter I followed by a number. Where reference is made below to incubator embodiments or also to I embodiments, these embodiments are meant.
I1. CO₂ incubator comprising a
usable space (11),
a pressure equalization opening (3) designed to adjust the pressure in the usable space
(11) to the ambient pressure, and a CO₂ supply
   (2),
   wherein the CO₂ incubator is designed to reduce the CO₂ concentration in the usable space
(11).
   I2. Incubator according to the preceding incubator embodiment, wherein the incubator is designed to deliver supply air to the usable space (11) in a controlled manner.
   I3. Incubator according to one of the preceding incubator embodiments, wherein the incubator is designed to actively introduce supply air into the usable space (11).
   I4. Incubator according to the incubator embodiment before last, wherein the incubator is designed to actively extract air from the usable space (11) and thereby deliver supply air via the pressure equalization opening (3).
   I5. Incubator according to one of the preceding incubator embodiments, wherein the pressure equalization opening (3) comprises a pressure equalization filter (32), which preferably comprises a sintered filter.
   6. Incubator according to one of the preceding incubator embodiments, wherein the incubator comprises a pump (41).
   I7. Incubator according to the preceding incubator embodiment, unless referred to in embodiment I3, wherein the pump (41) is designed to extract air from the usable space (11).

It is understood that the term “air” in this context refers to the gas and/or aerosol mixture that is present in the usable space.
I8. Incubator according to the preceding incubator embodiment, wherein an inlet of the pump (41) is fluidically connected to the usable space (11).
I9. Incubator according to one of the preceding incubator embodiments with the features of embodiment I4, wherein the incubator comprises an air outlet valve which is fluidically connected to the usable space (11) and an air outlet reservoir and is arranged between these, wherein the air outlet reservoir has a lower pressure than the usable space.
I10. Incubator according to one of the preceding incubator embodiments with the features of embodiment I6, unless referring back to embodiment I4, wherein the pump (41) is designed to convey supply air into the usable space (11).
I11. Incubator according to the preceding incubator embodiment, wherein an outlet of the pump (41) is fluidically connected to the usable space.
I12. Incubator according to one of the two preceding incubator embodiments, wherein the incubator further comprises a supply air filter (43) arranged downstream of the pump (41).
I13. Incubator according to one of the three preceding incubator embodiments, wherein an inlet of the pump (41) is connected to a supply air reservoir.
I14. Incubator according to one of the preceding incubator embodiments, wherein the incubator comprises an air duct (5), wherein the air duct comprises (5):
an inlet and an outlet, which are in each case connected to the usable space (11),
a fan (52) designed to convey air from the inlet to the outlet of the air duct (5), and
a flow resistance arranged upstream of the fan (52) and downstream of the inlet;
wherein a supply air duct (44) connected to a supply air reservoir is arranged between the flow resistance and the fan (52).
I15. Incubator according to the preceding incubator embodiment, wherein the air duct is designed in such a way that a negative pressure can be generated between the flow resistance and the fan (52).
I16. Incubator according to the preceding incubator embodiment, wherein the negative pressure may be at least up to 0.5 mbar, preferably at least up to 1 mbar below atmospheric pressure.
I17. Incubator according to one of the three preceding incubator embodiments, wherein the flow resistance is an air duct filter (53), preferably a particulate filter, and more preferably a HEPA filter.
I18. Incubator according to one of the four preceding incubator embodiments, wherein a supply air filter (43) is arranged in the supply air duct (44).
I19. Incubator according to one of the four preceding incubator embodiments, wherein a supply air valve (45) is arranged in the supply air duct (44).
I20. Incubator according to the preceding incubator embodiment and with the features of embodiment I18, wherein the supply air valve (45) is arranged downstream of the supply air filter (43).
I21. Incubator according to one of the two preceding incubator embodiments, wherein the supply air valve (45) is a proportional valve.
I22. Incubator according to one of the eight preceding incubator embodiments, wherein the air duct (5) comprises a humidifier (51) arranged upstream of the flow resistance (53).
I23. Incubator according to one of the preceding incubator embodiments, wherein the incubator comprises a CO₂ sensor designed to determine the CO₂ concentration in the usable space (11).
I24. Incubator according to one of the preceding incubator embodiments, wherein the incubator comprises a controller.
I25. Incubator according to the preceding incubator embodiment, wherein the controller is designed to reduce the CO₂ concentration in the usable space (11) if a CO₂ limit value is exceeded.
I26. Incubator according to the two preceding incubator embodiments, wherein the controller is designed to control and/or regulate the CO₂ concentration in the usable space (11) with respect to a predetermined setpoint.
I27. Incubator according to one of the preceding incubator embodiments and with the features of embodiments I19 and I24, wherein the controller is designed to control and/or regulate the supply air valve (45) and as an option also the fan (52).
I28. Incubator according to one of the preceding incubator embodiments and with the features of embodiment I6, wherein the pump is an air pump or a vacuum pump
I29. Incubator according to one of the preceding incubator embodiments and with the features of embodiments I6 and I24, wherein the controller is designed to control and/or regulate the pump.
I30. Incubator according to one of the preceding incubator embodiments and with the features of at least one of embodiments I12 or I18, wherein the supply air filter is a membrane filter.
I31. Incubator according to one of the preceding incubator embodiments and with the features of at least one of embodiments I12 or I18, wherein the supply air filter is a sterile filter, preferably made of PTFE.
I32. Incubator according to one of the preceding incubator embodiments, wherein the CO₂ supply (2) comprises a CO₂ valve (22) and a CO₂ filter (23) and is designed to be connected to a CO₂ reservoir (21).
I33. Incubator according to the preceding incubator embodiment, wherein the CO₂ filter (23) is arranged upstream of the CO₂ valve (22).
I34. Incubator according to one of the two preceding incubator embodiments, wherein the CO₂ valve is a proportional valve.
I35. Incubator according to one of the three preceding incubator embodiments and with the features of embodiment 16, wherein the incubator is designed in such a way that the pump does not operate if the CO₂ valve is open.
I36. Incubator according to one of the four preceding incubator embodiments, wherein the CO₂ filter (23) is a membrane filter.
I37. Incubator according to one of the five preceding incubator embodiments, wherein the CO₂ filter (23) is a sterile filter, preferably made of PTFE.
I38. Incubator according to one of the preceding incubator embodiments, wherein the supply air is at least one of the recirculating air of the incubator, oxygen, and/or nitrogen.
I39. Incubator according to one of the preceding incubator embodiments and with the features of I13 or I14, wherein the supply air reservoir is provided by the environment of the CO₂ incubator.

Reference is made below to method embodiments. These embodiments are identified by a letter V followed by a number. Where reference is made below to method embodiments or also to V embodiments, these embodiments are meant.
V1. Method for changing a CO₂ concentration in a usable space of a CO₂ incubator, wherein the method comprises:
Determination of the CO₂ concentration in the usable space,
Determination of the CO₂ concentration with a predetermined CO₂ value,
Reduction in the CO₂ concentration in the usable space of the CO₂ incubator if the CO₂ value is exceeded.
V2. Method according to the preceding method embodiment, wherein the predetermined CO₂ value is a CO₂ limit value.
V3. Method according to the method embodiment before last, wherein the predetermined CO₂ value is a CO₂ setpoint.
V4. Method according to one of the preceding method embodiments, wherein the determination of the CO₂ concentration comprises measurement of the CO₂ concentration in the air inside the CO₂ incubator.
V5. Method according to one of the preceding method embodiments, wherein reduction of the CO₂ concentration in the usable space comprises actively delivering supply air into the usable space.
V6. Method according to the preceding method embodiment, wherein the active delivery of supply air comprises extracting air from the usable space in order to allow supply air to enter, for example via a pressure equalization opening.
V7. Method according to the method embodiment before last, wherein the active delivery of supply air comprises pumping supply air into the usable space.
V8. Method according to one of the preceding method embodiments and with the features of method embodiment V5, wherein the active delivery of supply air comprises the suction of supply air via a supply air duct connected to an air duct of the incubator via negative pressure.
V9. Method according to one of the preceding method embodiments and with the features of method embodiment V5, wherein the method further comprises filtering of the supply air.
V10. Method according to one of the preceding method embodiments and with the features of method embodiment V5, wherein the supply air is delivered from a supply air reservoir.

The supply air reservoir may for example be the surrounding environment of the CO₂ incubator, with the result that the supply air corresponds to the ambient air. Alternatively, the supply air reservoir may also be provided by a container, e.g. a gas cylinder, meaning that the composition of the supply air can be controlled precisely.
V11. Method according to any of the preceding method embodiments, wherein the method comprises:
Increasing the CO₂ concentration in the usable space of the CO₂ incubator if the CO₂ value falls below the setpoint.
V12. Method according to one of the preceding method embodiments, wherein the method comprises receiving the CO₂ value.
V13. Method according to one of the preceding embodiments, wherein the method comprises controlling or regulating the CO₂ concentration as a function of the CO₂ value.
V14. Method according to the preceding method embodiment and with the features of method embodiment V11, wherein controlling the CO₂ concentration comprises determining, comparing, increasing, and reducing the CO₂ concentration in the usable space.
V15. Method according to one of the preceding method embodiments, wherein the CO₂ incubator is a CO₂ incubator according to one of the preceding incubator embodiments.
I40. Incubator according to one of the preceding incubator embodiments, wherein the incubator is designed to implement the method according to one of the preceding method embodiments.
I41. Incubator according to one of the preceding incubator embodiments and with the features of I24, wherein the controller is designed to implement the method according to one of the preceding method embodiments.

Embodiments of the present invention will now be described with reference to the accompanying drawings. These embodiments are only intended to exemplify, but not limit, the present invention.

Embodiments of the present invention will now be described with reference to the accompanying drawings. These embodiments are only intended to exemplify, but not limit, the present invention.

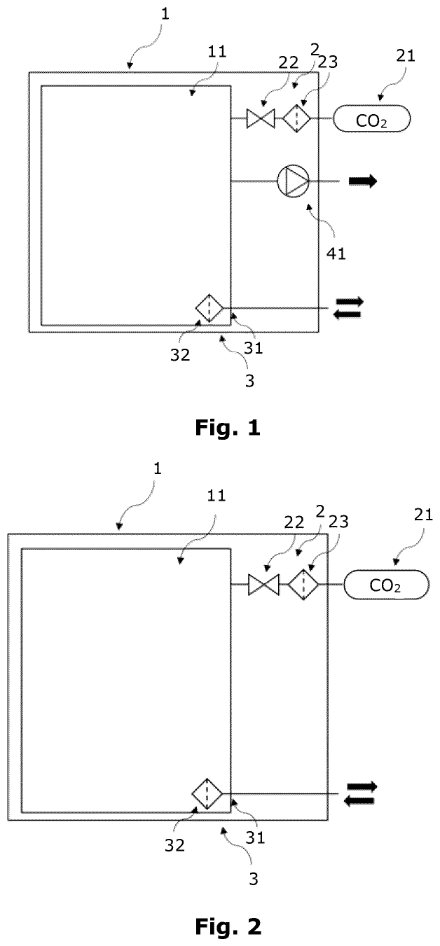

FIG. 1 shows an embodiment of a CO₂ incubator as per the invention;

FIG. 2 shows an embodiment of a CO₂ incubator as per the invention wherein air can be actively extracted from the usable space;

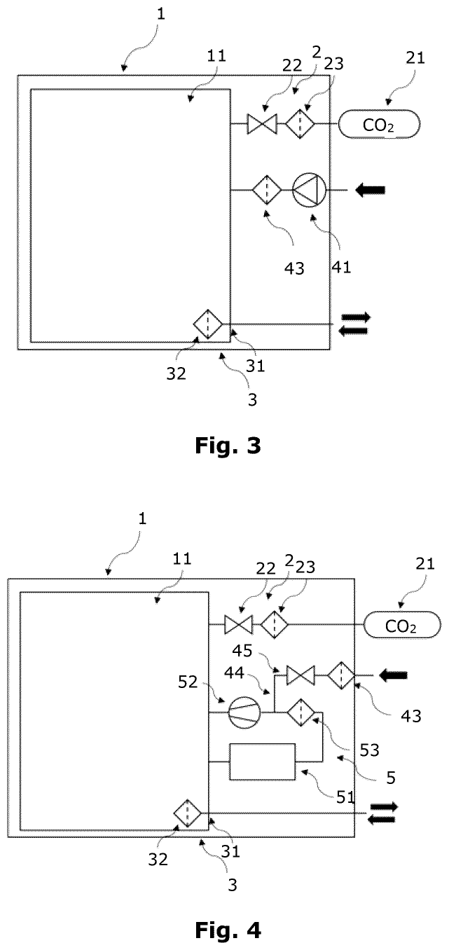

FIG. 3 shows an embodiment of a CO₂ incubator as per the invention wherein air can be actively conveyed to the usable space;

FIG. 4 shows a further embodiment of a CO₂ incubator as per the invention wherein air can be actively conveyed to the usable space; and

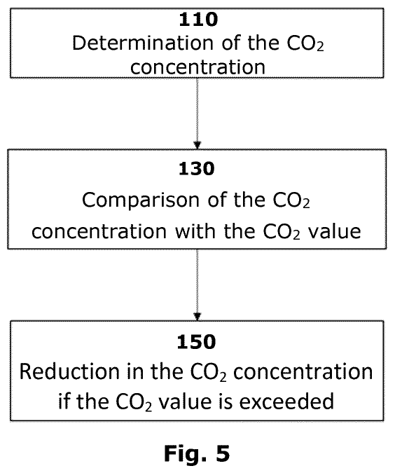

FIG. 5 illustrates a method as per the invention for changing the CO₂ concentration in the usable space of a CO₂ incubator.

It is noted that not all drawings bear all reference signs. Instead, in some of the drawings, some of the reference signs have been omitted for brevity and ease of presentation. Embodiments of the present invention will now be described with reference to the accompanying drawings.

With reference to FIG. 1, the present invention relates to a CO₂ incubator 1 (also referred to in the following simply as incubator 1), which comprises a usable space 11, a CO₂ supply 2, and a pressure equalization opening 3, wherein the pressure equalization opening 3 is designed to adjust the pressure in the usable space 11 to an ambient pressure of the incubator 1. In general, the CO₂ incubator 1 is designed to reduce the CO₂ concentration in usable space 11.

Usable space 11 is typically accessible via at least one door of incubator 1 and can be designed as a simple chamber. Containers with cells can then for example be placed in usable space 11, in order to expose these to controlled environmental or ambient conditions that can e.g. promote cell growth.

CO₂ supply 2 can be designed to be connected to a CO₂ reservoir 21, e.g. a gas cylinder filled with CO₂. CO₂ reservoir 21 can be designed to provide CO₂ at a specific pressure, e.g. at a maximum of 1 bar above atmospheric pressure. CO₂ reservoir 21 can for instance also be provided via a corresponding supply line. In some embodiments, the CO₂ reservoir may also be encompassed by incubator 1.

CO₂ supply 2 may comprise a CO₂ valve 22, which can optionally open or close CO₂ reservoir 21 and usable space 11. In particular, CO₂ valve 22 may preferably be a controllable or adjustable valve. CO₂ valve 22 may also preferably assume partially open positions in addition to an open and a closed position in order to control or regulate the CO₂ quantity and/or the pressure as a result. In other words, CO₂ valve 22 may be a proportional valve. A CO₂ filter 23 may also be provided. This filter may preferably be designed to filter any CO₂ that is introduced. In particular, contaminants in the form of particles as well as other materials, e.g. oils from the supply lines, can be removed from the gas stream and expelled. CO₂ filter 23 can for instance be designed as a membrane filter or as a sterile filter made of PTFE.

Pressure equalization opening 3 comprises a fluidic connection 31 between the usable space and the environment of the incubator or alternatively a reservoir maintained at ambient pressure. This fluid connection 31 can for instance be provided via a corresponding opening or a conduit. In addition, pressure equalization opening 3 preferably comprises a pressure equalization filter 32. Pressure equalization filter 32 may preferably be a sintered filter. The sintered filter may have a relatively large pore size, e.g. approx. 200 μm. Pressure equalization filter 32 essentially acts as a flow resistance. If therefore CO₂ is admitted to usable space 11 via CO₂ supply 2, the pressure in the usable space increases on an intermittent basis—for example to 1.3 atm, while the ambient pressure is 1.0 atm. Gas is released in this case from usable space 11 to the environment via pressure equalization opening 3, wherein the current gas flow between usable space 11 and the environment depends on the pressure difference between usable space 11 and the environment. As an advantage, pressure equalization filter 32 prevents any uninhibited and uncontrolled air exchange between the usable space and the environment—since it acts as a flow resistance, the gas exchange is lower than would be the case if no pressure equalization filter 32 were provided. If the pressure in the usable space is increased or decreased, a greater exchange with the ambient air can be achieved accordingly. The pressure equalization filter 32 may be designed to filter dust and coarse contaminants from the air flowing through.

Although an incubator is principally not completely airtight, meaning that a certain amount of air exchange could take place even without a corresponding pressure equalization opening 3, this air would, however, be unfiltered and there would be a high risk e.g. of unwanted condensation forming at the incubator door.

If CO₂ is supplied to usable space 11 via the CO₂ supply, i.e. particularly if the CO₂ valve 22 is opened, the pressure in usable space 11 is increased initially relative to the ambient pressure. Overall, this enables the CO₂ concentration in usable space 11 to be increased, as CO₂ is introduced and a mixture of air and/or gases comprising all gases (and aerosols, if applicable) present in the usable space escapes from the usable space via pressure equalization opening 3.

If the CO₂ valve 22 is closed, the pressure inside usable space 11 is equalized to the ambient pressure via pressure equalization opening 3. Any further air exchange between usable space 11 and the environment via pressure equalization opening 3 is inhibited by the flow resistance of the pressure equalization filter.

It is understood that the CO₂ incubator 1 according to the invention may additionally have other features. The incubator may in particular additionally be designed to control or regulate the temperature in the usable space. For this purpose, the incubator may for instance comprise a temperature control element and a temperature sensor. The incubator may also be designed to control or regulate the humidity in the usable space, e.g. via a humidification system 5.

It is understood that the incubator may also be designed to increase the CO₂ concentration in the usable space. The incubator may in particular be designed to control or regulate the CO₂ concentration in the usable space.

In general, the CO₂ incubator according to the invention is designed to reduce the CO₂ concentration in usable space 11. This can generally be achieved by actively delivering supply air to usable space 11, either by directly introducing supply air or by actively extracting air (or gas in general) from usable space 11, which is then replaced by the flow of supply air introduced. Corresponding embodiments are described below as examples. FIG. 2 shows an embodiment in which air can be actively removed from the usable space 11. A pump 41 may be provided for this purpose which can convey air from usable space 11 into the environment.

In the embodiment shown, the CO₂ concentration in usable space 11 can be increased by means of CO₂ supply 2 (as outlined above) on the one hand, and the CO₂ concentration in the usable space can be reduced using pump 41 on the other hand. For this purpose, pump 41 can convey air or gas from usable space 11 into the environment of CO₂ incubator 1. It is preferably provided that pump 41 only operates when CO₂ valve 22 is closed. The negative pressure created in usable space 11 in comparison to the surrounding environment is then counterbalanced via pressure equalization opening 3, via which supply air (preferably ambient air) flows into the usable space. This has the advantage of enabling a reduction in the CO₂ concentration in the usable space, provided that the CO₂ concentration in the supply air is below the CO₂ concentration in the usable space, and below the desired setpoint value when controlled accordingly. For instance, the CO₂ content in the atmosphere is approximately 0.04% or 400 ppm, but in closed rooms this may be higher depending on ventilation (e.g. up to 1000 ppm or even higher).

In other words, the CO₂ concentration can be reduced by extracting air or gas from the incubator, and more precisely from usable space 11 of the incubator. This can take place using a (vacuum/air) pump 41. Alternatively, the usable space can also be connected to an area with negative pressure via a valve in order to thereby extract air from the usable space. Pump 41 or the valve can be controlled by an electronic comparison of the setpoint/actual value or triggered if a CO₂ limit value is exceeded. Exceeding a CO₂ limit value may for instance trigger a CO₂ alarm.

Alternatively, usable space 11 can also be fluidically connected via an air outlet valve to an air outlet reservoir that has a lower pressure than the ambient pressure. If the air outlet valve is opened, air consequently flows from the usable space into the air outlet reservoir.

FIG. 3 shows a further embodiment wherein supply air can be actively conveyed into usable space 11. A pump 41 may be provided for this purpose, which conveys air from a supply air reservoir into the usable space. The supply air reservoir may preferably be provided by the environment of CO₂ incubator 1 or by a gas reservoir, e.g. a gas cylinder. This can enable ambient air or alternatively a gas or gas mixture with a controlled composition, e.g. consisting of nitrogen and oxygen, to be conveyed into the usable space. Similar to the embodiment shown in FIG. 2, it is preferably provided that pump 41 only operates if CO₂ valve 22 is closed.

The excess pressure arising in usable space 11 in comparison to the surrounding environment is then equalized via pressure equalization opening 3, via which air or gas escapes from the usable space into the surrounding environment. This has the advantage of enabling a reduction in the CO₂ concentration in the usable space, provided that the CO₂ concentration in the supply air is below the CO₂ concentration in the usable space, and below the desired setpoint value when controlled accordingly.

In order to prevent contaminants from the supplied air, an air filter 43 may also be provided, which filters the supplied air preferably downstream of pump 41. Supply air filter 43 may for example comprise a sterile and/or membrane filter. The advantage with this is that it can help to prevent contaminants in the usable space through the supplied air.

In other words, supply air is blown into the incubator, and more precisely into usable space 11. An (air) pump 41 is used for this which pumps air from outside the CO₂ incubator into usable space 11 via an air supply filter 43, which purifies the supply air. The pump can be triggered if a CO₂ limit value (e.g. a CO₂ alarm) is exceeded or can be or controlled by an electronic comparison of the setpoint/actual value.

FIG. 4 shows a further embodiment wherein supply air can be actively conveyed into usable space 11. In this embodiment, the supply air is introduced if needed via an air duct 5 encompassed by the CO₂ incubator.

Air duct 5 may generally be used for circulation and optional humidification of the air in the usable space. The air duct may comprise an inlet and an outlet for this purpose, each of which is connected to the usable space, and a fan 52 (also referred to as cooling fan 52) designed to convey air from the inlet to the outlet of air duct 5.

Air duct 5 may furthermore comprise a flow resistance 53 arranged upstream of fan 52 and downstream of the inlet. Flow resistance 53 may preferably comprise a filter 53, preferably a particulate filter 53, e.g. a HEPA filter. Air resistance that is provided by flow resistance 53 results in a pressure drop via flow resistance 53 (the pressure in front of, i.e. upstream of flow resistance 53 is therefore higher than the pressure behind, i.e. downstream of flow resistance 53). With a corresponding design for fan 52, a negative pressure can thus arise between fan 52 and flow resistance 53—the pressure between these units (i.e. downstream of resistance 53 and upstream of fan 52) is therefore lower than the pressure downstream of the fan. This negative pressure can be an advantage when used to introduce supply air into the usable space 11. The negative pressure can for instance be up to 1 mbar below atmospheric pressure.

For this purpose, a supply air duct 44 may be provided between flow resistance 53 and fan 52 via which supply air can be drawn in. Supply air duct 44 may advantageously comprise a supply air valve 45 which can open and close the supply air duct 44. Supply air valve 45 is a proportional valve which can also assume intermediate positions. Supply air duct 44 may furthermore also comprise a supply air filter 43, which filters the supplied air to prevent contamination of usable space 11.

Air duct 5 may comprise a humidifier 51 or nebulizer 51, which vaporizes and/or atomizes water from a water reservoir in order to increase the relative humidity in the usable space. Fan 52 and flow resistance 53 may preferably be arranged downstream of humidifier 51 and be designed to draw air from usable space 11 via humidifier 51 and blow this back into the usable space.

In other words, supply air may be drawn into usable space 11 through the incubator's own air duct system. This can be enabled by placing a flow resistance (preferably a particulate filter) 53, in particular a HEPA filter, in front of a fan 52, which draws air from usable space 11 through the flow resistance 53, thereby creating a negative pressure between flow resistance 53 and fan 52. The negative pressure is caused by the pressure drop via HEPA flow resistance 53, which is proportional to the volume flow through the flow resistance and can range between 0 and −1 mbar (relative to the atmospheric pressure). In order to prevent contaminants or particles from entering the usable space, supply air can be drawn in via an external supply air filter 43 (e.g. a membrane filter) and for instance fed into a filter housing of particulate filter 53. The correct amount of air drawn in may be adjusted via a pressure drop in external air filter 43 and, if applicable, via an additional capillary tube in supply line 44. The air intake can be controlled and/or regulated by installing a supply air valve 45 between supply air filter 43 and the negative pressure region between flow resistance 53 and fan 52, e.g. the filter housing of filter 53 provided for instance by a hose connected to the HEPA filter airbox. The air intake may for instance be triggered by exceeding a CO₂ limit value (which may trigger a CO₂ alarm) or controlled by an electronic comparison of the setpoint/actual value.

This embodiment advantageously utilizes an air duct 5 that typically already exists in any case (e.g. as part of a humidification system), thereby allowing an additional pump 41 to be omitted compared to the embodiments shown in FIGS. 2 and 3. Compared to using an additional pump, however, only a small volume of air can be introduced per unit of time, whereas an additional pump 41 can deliver larger volumes per unit of time.

The supply air can advantageously be ambient air of incubator 1, i.e. any supply air reservoirs can be provided by the environment of the CO₂ Incubator. Alternatively, supply air reservoirs that contain a predetermined gas or gas mixture can also be used.

The CO₂ incubator may generally include a controller that can monitor the CO₂ concentration in the usable space, e.g. using a corresponding CO₂ sensor, and can regulate or preferably control this if necessary. For this purpose, the controller can exchange data with at least one CO₂ sensor, CO₂ valve 22, and depending on the design of pump 41, the supply air valve 45 and/or fan 52 in order to read out data and/or regulate or control this. In this way, the controller can initiate measures to reduce the CO₂ concentration, e.g. if a CO₂ limit value is exceeded, and trigger a CO₂ alarm if necessary, or alternatively can actively control the CO₂ concentration via a comparison of the actual value with the setpoint.

With reference to FIG. 5, the present invention also relates to a corresponding method for changing the CO₂ concentration in the usable space of a CO₂ incubator. This method involves determining the CO₂ concentration in the usable space, comparing the CO₂ concentration with a predetermined CO₂ value, and reducing the CO₂ concentration in the usable space of the CO₂ incubator if the CO₂ value is exceeded.

The CO₂ value may be a CO₂ limit value or a CO₂ setpoint in this context. The CO₂ limit value can be used to detect excessively high CO₂ concentrations, trigger a CO₂ alarm if necessary, and initiate appropriate reduction measures directly and automatically. The CO₂ setpoint allows the CO₂ concentration to be controlled by comparing it with an actual value for the CO₂ concentration in the usable space determined by a CO₂ sensor.

The method may involve receiving the CO₂ value. The CO₂ value may for instance be predetermined by a user.

The step of reducing the CO₂ concentration may involve actively delivering supply air to the usable space, either by extracting air or gas from the usable space, meaning that supply air flows in e.g. via a pressure equalization opening, or by pumping supply air into the usable space. Alternatively, supply air can also be introduced from a supply air reservoir via a valve in a similar way to CO₂, wherein the supply air reservoir provides supply air at a pressure that is higher relative to the usable space.

The method may also involve increasing the CO₂ concentration in the usable space if the CO₂ value falls below the setpoint. As described above, an increase in the CO₂ concentration can be achieved by supplying CO₂ from a CO₂ reservoir that can provide CO₂ at a higher pressure relative to the usable space, for instance by opening a CO₂ valve arranged between the usable space and the CO₂ reservoir.

The method may in particular involve regulating and preferably controlling the CO₂ concentration as a function of the CO₂ value, wherein controlling the CO₂ concentration involves determining, comparing, increasing, and reducing the CO₂ concentration in the usable space.

The present invention thus advantageously enables reduction of excessively high CO₂ concentrations in the incubator automatically, thereby avoiding error messages that are typically emitted by known devices. In particular, the users do not have to intervene themselves and, for example, open the door manually.

If a relative term such as “about,” “substantially,” or “approximately” is used in this description or the claims, any such term should also be construed as including the precise term. That is to say, for example, “substantially straight” should be construed as also including “(exactly) straight.”

Whenever steps were recited in the claims above or also in those appended, it should be noted that the order in which the steps are recited in this text may be random. This means the order in which the steps are recited may be random unless otherwise specified or obvious to a person skilled in the art. So if in the present document, for example, it is stated that a method comprises steps (A) and (B), this does not necessarily mean that step (A) occurs before step (B), but it is also possible that step (A) (at least in part) is performed simultaneously with step (B) or that step (B) occurs before step (A). Furthermore, if it is stated that a step (X) precedes another step (Z), this does not mean that there is no step between steps (X) and (Z). This means step (X) before step (Z) comprises the situation where step (X) is performed directly before step (Z), but also the situation where (X) is performed before one or more steps (Y1), . . . , followed by step (Z). Corresponding considerations apply when terms such as “after” or “before” are used.

While a preferred embodiment has been described above with reference to the accompanying drawings, the person skilled in the art will understand that this embodiment was provided for illustration only and should by no means be construed as limiting the scope of the present invention, which is defined by the claims.

1. A CO₂ incubator, comprising:
a usable space (11);
a pressure equalization opening (3) designed to adjust the pressure in the usable space (11) to an ambient pressure; and
a CO₂ supply (2), wherein the CO₂ incubator is designed to reduce any CO₂ concentration in the usable space (11).

2. The incubator of claim 1, wherein the pressure equalization opening (3) comprises a pressure equalization filter (32), which pressure equalization filter optionally comprises a sintered filter.

3. The incubator of claim 1, wherein the incubator is designed to actively extract air from the usable space (11) and thereby deliver supply air via the pressure equalization opening (3).

4. The incubator of claim 1, wherein the incubator comprises a pump (41) and wherein the pump (41) is designed to extract air from the usable space (11).

5. The incubator of claim 1, wherein the incubator is designed to actively introduce supply air into the usable space (11).

6. The incubator of claim 1, wherein the incubator comprises a pump (41) and wherein the pump (41) is designed to convey supply air into the usable space (11).

7. The incubator of claim 1, wherein the incubator comprises an air duct (5), wherein the air duct (5) comprises:
an inlet and an outlet, which are in each case connected to the usable space (11),
a fan (52) designed to convey air from the inlet to the outlet of the air duct (5), and a flow resistance (53) arranged upstream of the fan (52) and downstream of the inlet;
wherein a supply air duct (44) connected to a supply air reservoir is arranged between the flow resistance (53) and the fan (52).

8. The incubator of claim 1, wherein the incubator comprises a controller, and wherein the controller is designed to;
reduce the CO₂ concentration in the usable space (11) if a CO₂ limit value is exceeded, and/or
control and/or regulate the CO₂ concentration in the usable space (11) with respect to a predetermined setpoint.

9. A method for changing a CO₂ concentration in a usable space of a CO₂ incubator, comprising:
determining a CO₂ concentration in the usable space,
comparing the CO₂ concentration with a predetermined CO₂ value, and
reducing the CO₂ concentration in the usable space of the CO₂ incubator if the predetermined CO₂ value is exceeded.

10. The method of claim 9, wherein reducing the CO₂ concentration in the usable space involves actively conveying supply air into the usable space.

11. The method of claim 10, wherein actively conveying supply air comprises suction of supply air in via a supply air duct connected to an air duct of the incubator via negative pressure.

12. The method of claim 9, further comprising increasing
the CO₂ concentration in the usable space of the CO₂ incubator if a CO₂ value falls below a setpoint.

13. The method of claim 9, further comprising regulating the CO₂ concentration as a function of a CO₂ value, and wherein controlling the CO₂ concentration comprises determining, comparing, increasing, and reducing the CO₂ concentration in the usable space.
